(19) United States
(12) Patent Application Publication
(10) Pub. No.: US 2006/0281121 A1
(43) Pub. Date: Dec. 14, 2006

(54) Computing with biomolecules

(76) Inventors: Ron Unger, Rechovot (IL); John Moult, Rockville, MD (US)

Correspondence Address: PRTSI, Inc., P.O. Box 16446, Arlington, VA, 22215, US

(21) Appl. No.: 11/448,762

(22) Filed: Jun. 8, 2006

Related U.S. Application Data
(60) Provisional application No. 60/688,329, filed on Jun. 8, 2005.

(51) Int. Cl. H01L 29/08; C12Q 1/68; C12M 1/34; H01L 35/24; H01L 51/00

(52) U.S. Cl. 435/6; 435/287.2; 977/702; 977/924; 257/40

(57) ABSTRACT

The present invention is of a molecular entity comprising a polypeptide core and nucleic acid sequences attached thereto which is capable of forming a universal logic gate such as the NAND gate. Specifically, the present invention can be used to device a molecular computing unit which can be used to detect biological markers and administer therapeutic moieties.

### RELATED APPLICATION

This application claims the benefit of U.S. Provisional Patent Application No. 60/688,329, filed on Jun. 8, 2005, the content of which is hereby incorporated by reference.

### FIELD AND BACKGROUND OF THE INVENTION

The present invention relates to a molecular computing unit and, more particularly, to methods of generating and using same in various biological implications.

In recent years there has been significant interest in exploring the possibilities of biological computation. A large number of studies have investigated various ideas for using biological molecules to carry out various types of calculations and computations (1-6).

Biological systems perform computations in living organisms on multiple levels, from the cognitive to the molecular. Examples range from the brain's ability to perform numerical calculations or analyze images to the immune system's ability to identify intruders. Other cellular activities, such as maintaining homeostatic levels of vital parameters and controlling expression levels of genes, are also forms of computation.

In contrast to these natural processes, the phrase “biological computation” usually suggests the use of biological molecules to carry out a general-purpose computation, i.e., a computation that can be considered to be a digital computation outside the realm of the biological world. One of the ultimate goals is to build a computer, quite similar in its basic operation to current silicon-based machines, with its underlying hardware (or better said “wetware”) based on biological components.

Considering the superb performance of silicon-based computers, one can question the need for biological alternatives. The advantages of a biological computer might be related to smaller size (Angstroms vs. microns), much lower energy consumption (e.g., the energy consumption in current supercomputers is more than 10⁻⁹ joule per operation), and ease of production of the components by genetic engineering. However, biological systems have significant disadvantages compared to silicon-based systems including slower speed of computation (GHZ for silicon-based computers compared with microseconds to milliseconds for biological reactions), durability (most biological components have limited half-lives), and reliability (most biological reactions are prone to a non-negligible error rates).

Thus, it is reasonable to suggest that the appropriate use of biological computational devices will be in environments where they naturally belong, for example in medicine where such devices can be encapsulated within a semi-permeable membrane, and installed inside a living body. In such a device, inputs might be biological signals and the output might trigger biological processes. A biological device would also have the significant advantage of being able to use internal energy resources, like ATP molecules, rather than being dependent on external or rechargeable energy sources. An example might be an insulin regulation system, where the input would reflect glucose levels and oxygen demand, and the output would be used to trigger insulin production on-site. Such a biological system may offer several advantages over current continuous pump systems, which are based on standard electronics.

Most of the current studies of biological computation have focused on DNA based systems. In such systems, the underlying computational element is the hybridization of single stranded DNA molecules to a complementary strand with high specificity. The computational paradigm takes advantage of the huge number of available DNA molecules to carry out, in effect, a parallel exhaustive search of the solution space. This idea originated with the pioneering study of Adelman (1) on solving the Hamiltonian path problem. It has been shown to work on other NP-hard computational problems like the Maximal Clique problem (Ouyang et al., 1997) and the 3-SAT problem (3) which is the archetype of the NP-hard problems, for which no efficient polynomial time algorithm is likely. These studies clearly demonstrated that DNA based computations are feasible. Nevertheless, these methods require the use of an exponential number (in the size of the problem) of molecules since their mode of operation is based on “a massive parallel attack”. Namely, each molecule represents another solution and all the solutions are tested simultaneously by a biological process (e.g., running all DNA molecules on an electrophoresis gel to isolate the shortest one). While this exponential dependency may be unavoidable in dealing with NP-hard problems, it will lead to a very inefficient solution to more tractable problems, where a more direct and efficient approach might be more appropriate. In addition, these systems require specific encoding and implementation for each problem, and thus, in a practical sense, they do not offer a way of utilizing such procedures as a generic way to solve general computational problems.

In an advance from DNA-only based computation, Shapiro and co-workers (4, 5), demonstrated how a finite automation can be built from restriction enzymes and ligases working on input presented as double stranded DNA. The automation was able to distinguish between strings with an odd versus an even number of input symbols. The computational devices described in (4, 5) are finite automata. The authors consider this as a first step towards building a Turing machine based on biological components. A Turing machine (8) is a general computational device which is the abstraction of all other known digital computational devices. While the model and its biological implementation are elegant, Turing machines are not efficient computational devices, and “programs” written for Turing machines are long and cumbersome.

Recently, these authors (6) have demonstrated that their approach can be used in a biological and medical set-up when they design a system where the inputs are MRNA molecules which are marker for diseases. After a digital computation which depends on the input, the output of the system is the production of a single strand DNA molecule with therapeutic effects.

Various other possibilities for biological devices for digital computing have been explored. One direction is focused on designing biological wires. In (9) it was demonstrated that silver plated DNA strands can be used as conducting wires. RecA was used (10) to bind to DNA in a sequence-dependent manner and thus control the conductivity patterns of DNA molecules. This approach may lead to a “hard wire” (or “wet wire”) form of biological computing. Nevertheless, such a system will depend on a conventional power supply and regular electronic switching devices.

There is at least one well-described natural example in which biological reactions are used to achieve a switching effect: In the chemotaxis system, phosphorylation and methylation were shown to work together to achieve a switching effect on bacterial mobility (11). This system is composed of several proteins with sophisticated feed-back mechanisms. Recently, attention was drawn to demonstrating that switching networks can be designed and engineered. A significant achievement in this direction is described in (12) where three transcriptional repressor systems were used to create an artificial oscillating network in E. coli. The network periodically, typically with periods of hours, triggered the synthesis of green fluorescent protein as a single cell read-out of its state. In (13) a toggle switch was constructed from two repressible promoters arranged in a mutually inhibitory network. The switch can be flipped sharply between stable states using transient chemical or thermal stimuli.

Several possibilities for an elementary biology-based switching unit have been explored. One scheme uses rhodopsin molecules and their ability to change conformation in response to light. Such molecules have been shown to be particularly useful in building biological memory elements (14). Another possibility that has been explored is to use a modified form of Ribonuclease A, in which the molecular switch is constructed from a non-natural amino-acid side chain, containing an electron donor group and an electron acceptor group, connected to one another with a conjugated double bond bridge. The switching mechanism is based on azonium-hydrazo tautomerization, by which a charge separation induced in the excited state causing a rearrangement of the electronic structure of the molecule, resulting in the exchange of locations of single and double bonds. This rearrangement of bonds leads to different three-dimensional conformations of the switch, one of which blocks access to the enzyme active site, effectively providing an on/off switch (15,16). While this switch design is very elegant, it is not clear how such elements can be hooked together to form a computing network.

Bray et al., (17) pointed out the diversity of roles proteins play in processing information in living cells, and suggested various possibilities for utilizing proteins to perform computational tasks.

However, to date a molecular computing unit which uses proteins to execute computational tasks guided using the universal logic gates has not been described.

There is thus a widely recognized need for, and it would be highly advantageous to have, a molecular computing unit devoid of the above limitations.

### SUMMARY OF THE INVENTION

According to one aspect of the present invention there is provided a molecular entity comprising a polypeptide core attached to at least two input nucleic acid sequences and at least one output nucleic acid sequence, wherein hybridization of the at least two input nucleic acid sequences with two complementary nucleic acid sequences modifies the at least one output nucleic acid sequence and optionally the polypeptide core.

According to another aspect of the present invention there is provided a composition-of-matter comprising a plurality of molecular entities, each of the plurality of molecular entities comprising a polypeptide core attached to at least two input nucleic acid sequences and at least one output nucleic acid sequence, wherein hybridization of the at least two input nucleic acid sequences with two complementary nucleic acid sequences of the plurality of molecular entities modifies the at least one output nucleic acid sequence and optionally the polypeptide core.

According to yet another aspect of the present invention there is provided a device comprising logic gates composed of the composition-of-matter.

According to still another aspect of the present invention there is provided a molecular entity comprising a polypeptide core attached to at least two nucleic acid sequences, wherein hybridization of the at least two nucleic acid sequences with two complementary nucleic acid sequences modifies the polypeptide core.

According to further features in preferred embodiments of the invention described below, the polypeptide core comprises at least two catalytic functions.

According to still further features in the described preferred embodiments the at least two catalytic functions comprise a kinase activity and an exonuclease activity.

According to still further features in the described preferred embodiments the polypeptide core comprises at least one monomer of at least one dimerizable polypeptide.

According to still further features in the described preferred embodiments at least one of the at least two catalytic functions comprises a kinase activity.

According to still further features in the described preferred embodiments the molecular entity capable of forming a logic gate.

According to still further features in the described preferred embodiments the logic gate is a NAND gate.

According to still further features in the described preferred embodiments the logic gate is a NOR gate.

According to still further features in the described preferred embodiments the logic gate is selected from the group consisting of the AND gate, the OR gate, the NOT gate, the XOR gate and XNOR.

According to still further features in the described preferred embodiments the kinase activity is a negatively regulated kinase.

According to still further features in the described preferred embodiments the negatively regulated kinase is DRP-1.

According to still further features in the described preferred embodiments the at least two input nucleic acid sequences are comprised in a single polynucleotide.

According to still further features in the described preferred embodiments the single polynucleotide further comprises the output nucleic acid sequence.

According to still further features in the described preferred embodiments each of the input nucleic acid sequences is double-stranded.

According to still further features in the described preferred embodiments the output nucleic acid sequence is double-stranded.

According to still further features in the described preferred embodiments the polypeptide core comprises a phosphatase activity.

According to still further features in the described preferred embodiments modification of the at least one output nucleic acid sequence comprises nucleic acid denaturation.

According to still further features in the described preferred embodiments modification of the polypeptide core comprises phosphorylation.

According to still further features in the described preferred embodiments the polypeptide core of each of the plurality of molecular entities is identical.

According to still further features in the described preferred embodiments the composition-of-matter further comprising ATP.

According to still further features in the described preferred embodiments a combination of three of the plurality of molecular entities is capable of forming a logic gate.

According to still further features in the described preferred embodiments the composition-of-matter is capable of forming at least two layers of logic gates.

The present invention successfully addresses the shortcomings of the presently known configurations by providing a molecular entity capable of forming a universal logic gate which can be used in a molecular computing unit.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c are schematic illustrations depicting a Boolean majority function expressed as a network of NAND gates. The output is true if at least two of the three inputs (A, B or C) are true. FIG. 1a depicts an example of a function expressed in terms of the binary NAND operations (N stands for NAND). FIG. 1b depicts the truth table of the function. For every combination of the input binary parameters A, B and C the function output value is shown under the M column. The boxed row depicts the computation for the instance logical circuit shown in FIG. 1c. FIG. 1c depicts a logical circuit, using NAND gates, that implements the function. Note that each input bit (either A, B or C) is fed-in into the network via more than one input gate. Such networks can be implemented by using protein complexes which function as NAND gates and DNA tags that function as connections between tags.

FIG. 2 is a schematic view of one embodiment of the computational element (also referred to as a “molecular entity” hereinafter) of the present invention. The molecular entity includes a protein molecule (also referred to as a “polypeptide core” hereinafter) and three nucleic acid sequences (DNA tags). The protein molecule has two enzymatic domains to facilitate activation and computation. For example, these domains can be an exo-nuclease domain (for activation) and a kinase domain (for computation). In addition, each protein molecule is connected to three DNA tags to provide recognition properties, two for the input (shown in lowercase letters, also referred to as “input nucleic acid sequence” hereinafter) and one for output (shown in capitol letters, also referred to as “output nucleic acid sequence” hereinafter). The output tag is initially blocked by a complementary oligomer (TAGT, shown in green), rendering it inactive until the appropriate stage of the computation.

FIGS. 3a-f schematically depict the activation of a gate complex (i.e., the composition-of-matter of the present invention which includes a plurality of the molecular entities). FIG. 3a—The process starts with two input molecules (input molecular entities), each including a protein molecule connected (e.g., conjugated) to active DNA tags (shown in lowercase letters), which defuse freely in search for the appropriate target molecule (target molecular entity) which includes a protein molecule connected to DNA tags that are complementary to the active DNA tags. In this example, one molecular entity includes the “aggt” active tag, and the other molecular entity includes the “ccga” active tag. Note that the output tag of the target molecule is blocked by a matching oligomer (“CCG”, also referred to as “a polynucleotide hybridizable to the output nucleic acid sequence” hereinafter), and that the molecule is set by default to the de-phosphorylated (white) (i.e., set to the ‘zero’) state. FIG. 3b—The tags of the input molecules hybridize to the input tags of the target molecule. FIG. 3c—Once the two input molecules are tethered to their target, their localization (as dictated by the DNA tags linked thereto) causes these molecules to form an active dimer. FIG. 3d—The conditional phosphorylation reaction, representing the NAND gate: Only if both input molecules are phosphorylated (in the ‘one’ state, red) is the target molecule not phosphorylated. In all other combinations, like the one shown here, the output molecule is phosphorylated. (i.e., set to the ‘one’ state—red). FIG. 3e—Formation of the complex (regardless of its phosphorylation state) activates the output tag as a result of exo-nuclease digestion of the blocking oligomer, exposing the single stranded output sequence. FIG. 3f—The output molecule (which is also a molecular entity including a protein molecule with DNA tags connected thereto) diffuses in search of its own target.

FIG. 4 is schematic view of recognition tags attached to the protein part of the active molecule. Two input recognition sequences are shown (red) separated by linker sequence (blue). The output tag is covered by a matching strand which blocks accessibility until an exo-nuclease is used to digest the cover. The exo-nuclease will only digest the DNA cover [i.e., the complementary oligonucleotide (the hybridizable polynucleotide, CCG in this case) bound to the end of the output tag] and will leave intact the DNA (or PNA) tag;

FIGS. 5a-c are graphs depicting the performance of the simulation a circuit based on NAND gates with a full binary tree structure. Running time is measured as the number of generations (a generation consists of moving every molecule once) until 50% of the output molecules are activated. FIG. 5a—Running time decreases exponentially in the diffusion rate, i.e., in the distance (in grid units) each element can move in one step. FIG. 5b—Running time increases exponentially in the depth of the circuit; FIG. 5c—Running time decreases exponentially in the number of the copies for each molecule.

### DESCRIPTION OF THE PREFERRED EMBODIMENTS

The present invention is of a molecular entity comprising a polypeptide core and nucleic acid sequences attached thereto which is capable of forming a universal logic gate such as the NAND gate. Specifically, the present invention can be used to device a molecular computing unit which can be used to detect biological markers and administer therapeutic moieties.

The principles and operation of the molecular entity according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Biological molecules have been suggested to carry out various types of calculations and computations in the form of biological computing units (1-6). The advantages of the suggested biological computing units relate to smaller size (Angstroms vs. microns), lower energy consumption and ease of production of the components by genetic engineering. In addition, biological computational devices can be encapsulated within a semi-permeable membrane, and installed inside a living body. In such a device, inputs might be biological signals and the output might trigger biological processes. A biological device would also have the significant advantage of being able to use internal energy resources, like ATP molecules, rather than being dependent on external or rechargeable energy sources. An example might be an insulin regulation system, where the input would reflect glucose levels and oxygen demand, and the output would be used to trigger insulin production on-site.

Attempts to generate biological computing units have focused on DNA based systems. In such systems, the underlying computational element is the hybridization of single stranded DNA molecules to a complementary strand with high specificity. For example, Shapiro and co-workers (4, 5), made the first step towards a Turing machine and demonstrated how a finite automation can be built from restriction enzymes and ligases working on input presented as double stranded DNA. However, Turing machines are not efficient computational devices, and “programs” written for Turing machines are long and cumbersome.

Other studies have focused on designing biological wires. In (9) it was demonstrated that silver plated DNA strands can be used as conducting wires. RecA was used (10) to bind to DNA in a sequence-dependent manner and thus control the conductivity patterns of DNA molecules. This approach may lead to a “hard wire” (or “wet wire”) form of biological computing. Nevertheless, such a system depends on a conventional power supply and regular electronic switching devices.

Bray et al., (17) pointed out the diversity of roles proteins play in processing information in living cells, and suggested various possibilities for utilizing proteins to perform computational tasks.

However, to date a molecular computing unit which mimics a universal logic gate has not been suggested or described.

While reducing the present invention to practice, the present inventors devised a molecular entity capable of functioning as a universal logic gate and therefore can be used to construct a biological computing unit. Since the biological computing unit of the present invention is based on the universal logic gates it can be used to execute any computational task by simple programming of the universal logic gates. Such a biological computing unit offers a useful combination of natural interface to biological processes with the strength of digital computation to achieve accuracy and precision.

Thus, according to one aspect of the present invention there is provided a molecular entity. The molecular entity of the present invention comprising a polypeptide core attached to at least two input nucleic acid sequences and at least one output nucleic acid sequence, wherein hybridization of the at least two input nucleic acid sequences with two complementary nucleic acid sequences modifies the at least one output nucleic acid sequence and optionally the polypeptide core.

The phrase “input nucleic acid sequences” refers to an isolated nucleic acid sequence which is capable of receiving an output signal from an output nucleic acid sequence by way of nucleic acid complementarity (e.g., hybridization).

The phrase “output nucleic acid sequence” refers to an isolated nucleic acid sequence which can be received as an input signal by way of nucleic acid complementarity to an input nucleic acid sequence.

Thus, it will be appreciated that sequence complementation between the output nucleic acid sequence and the input nucleic acid sequence may serve as a wire as further described hereinbelow.

It should be noted that since each of the input or output nucleic acid sequences is attached to a polypeptide core, hybridization of two output nucleic acid sequences of two distinct molecular entities to the two input nucleic acid sequences of another molecular entity may trigger the formation of a protein complex between the three polypeptide cores of the three molecular entities.

The term “attached” refers to direct or indirect (e.g., via a linker) attachment to the polypeptide core. Attachment can be achieved by covalent conjugation using methods known in the art such as those described in Bruick RK, et al., 1996 (19) and Example 4 of the Examples section which follows.

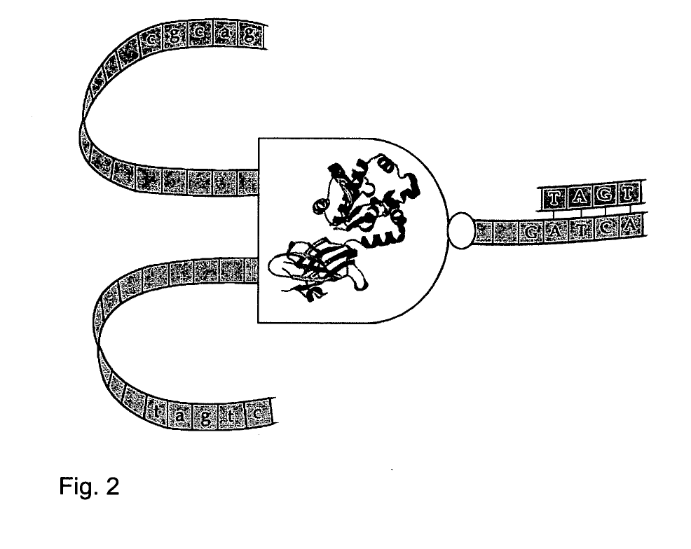

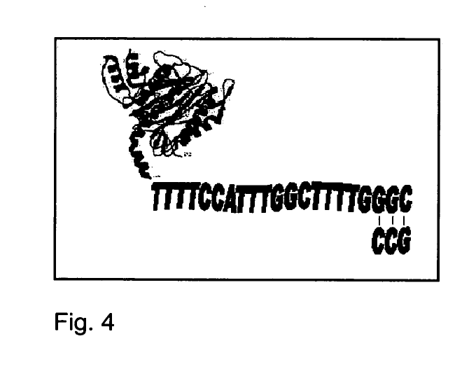

The input or output nucleic acid sequences can be connected to the polypeptide core in various configurations. For example, nucleic acid sequence segments (individual) can be attached to the polypeptide core. Alternatively, the input and/or output nucleic acid sequences can reside on a single contiguous nucleic acid sequence which is attached to the polypeptide core (for various configurations see FIGS. 2 and 4). It will be appreciated that any of the nucleic acid sequences used by the present invention can further include additional nucleic acid sequences which may serve as linkers. The sequence and length of such linkers are selected such that following hybridization between an input nucleic acid sequence of one molecular entity with an output nucleic acid sequence of another molecular entity the desired protein complexes may be formed. Thus, the order of the input/output nucleic acid sequences and/or the length of the linkers can control the formation of protein complexes between polypeptide cores of different molecular entities.

A nucleic acid sequence of this aspect of the present invention may be single stranded or double stranded oligomer or polymer of ribonucleic acid (RNA) or deoxyribonucleic acid (DNA) or mimetics thereof. These terms include natural DNA or RNA sequences or oligonucleotides composed of naturally-occurring bases, sugars and covalent internucleoside linkages (e.g., backbone) as well as oligonucleotides having non-naturally-occurring portions which function similarly to respective naturally-occurring portions.

Nucleic acid sequences designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Preferably used oligonucleotides are those modified in either backbone, intemucleoside linkages or bases, such as oligonucleotides having modified backbones that retain a phosphorus atom in the backbone (e.g., phosphorothioates) or those that do not include a phosphorus atom therein. These may be preferably used for enduring the degradation potential of physiological environments.

Other oligonucleotides which can be used according to the present invention, are those modified in both sugar and the intemucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to the nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

As is described in Example 2 of the Examples section which follows, the input or the output nucleic acid sequences can be either single-stranded or double-stranded, depending on their use. Thus, when the molecular entity is designed to be inactive, both input nucleic acid sequences and output nucleic acid sequences are double-stranded and therefore cannot hybridize with complementary sequences. On the other hand, while activated, the output nucleic acid sequence of the molecular entity becomes single-stranded (e.g., by the action of exonuclease) and the input nucleic acid sequences are double-stranded.

As used herein the phrase “polypeptide core” refers to a synthetic or naturally occurring protein or fraction thereof which is able to modify the at least one output nucleic acid sequence and optionally the polypeptide core, as further described hereinbelow.

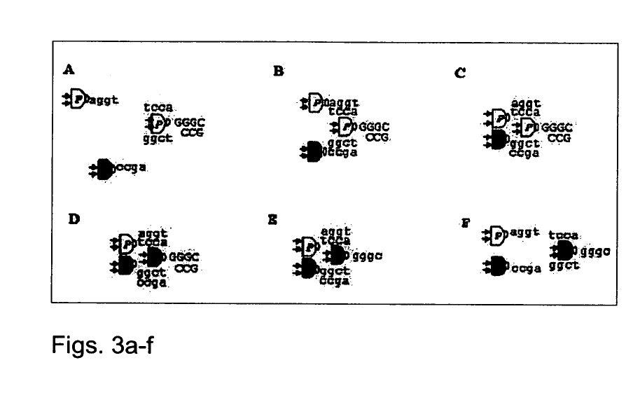

As is illustrated in FIGS. 3a-f and is described in Example 2 of the Examples section which follows, the polypeptide core comprises at least one monomer of at least one dimerizable (i.e., capable of forming a dimer) polypeptide. Such a dimerizable polypeptide can form a polypeptide dimer with another polypeptide core of another molecular entity. Preferably, the polypeptide dimer is a homodimer, i.e., composed of two identical monomers of polypeptide cores.

According to one preferred embodiment of the present invention, the catalytic activity of each monomer is depended on dimer formation. When such a dimer is formed, the catalytic activity of the two monomers can be activated.

For example, as is schematically shown in FIGS. 3d-e and described in Example 2 of the Examples section which follows, dimer formation results in activation of an exonuclease activity which removes an hybridizable polynucleotide which formed double stranded nucleic acid sequence with the output nucleic acid sequence. It will be appreciated that following the removal of the hybridizable polynucleotide the output nucleic acid sequence is single-stranded and thus can be recognized by another input nucleic acid sequence.

Preferably, the polypeptide core comprises at least two catalytic functions (e.g., enzymatic activities). Such catalytic functions can catalyze any chemical or biological reactions including covalent modifications and non-covalent modifications (e.g., formation/dissociation of hydrogen bonds). For example, the polypeptide core can include a kinase activity (i.e., which phosphorylates a substrate), a phosphatase activity (i.e., which removes phosphate from a substrate), an exonuclease activity (e.g., which degrades double-stranded or single-stranded nucleic acids), a methylase activity, an acetylase activity, a glycosylation activity, a hydrolase activity, as well as acylation, amidation, imitation and sulfation, and the enzymes that remove such modifications.

Preferably, the at least two catalytic functions comprise a kinase activity and an exonuclease activity. A non-limiting example of a polypeptide core is schematically illustrated in FIG. 2 and is described in Examples 2 and 4 of the Examples section which follows.

A description of natural and non-natural amino acids which can be used to generate the polypeptide core of the molecular entity of the present invention is provided in PCT Appl. No. IL2004/000744, which is fully incorporated herein by reference.

As used herein the term “mimetics” refers to molecular structures, which serve as substitutes for the peptide of the present invention in performing the biological activity (Morgan et al. (1989) Ann. Reports Med. Chem. 24:243-252 for a review of peptide mimetics). Peptide mimetics, as used herein, include synthetic structures (known and yet unknown), which may or may not contain amino acids and/or peptide bonds, but retain the structural and functional features of the peptide. The term, “peptide mimetics” also includes peptoids and oligopeptoids, which are peptides or oligomers of N-substituted amino acids [Simon et al. (1972) Proc. Natl. Acad. Sci. USA 89:9367-9371]. Further included as peptide mimetics are peptide libraries, which are collections of peptides designed to be of a given amino acid length and representing all conceivable sequences of amino acids corresponding thereto. Methods of producing peptide mimetics are described hereinbelow.

The polypeptide of present invention can be biochemically synthesized such as by using standard solid phase techniques. These methods include exclusive solid phase synthesis, partial solid phase synthesis methods, fragment condensation and classical solution synthesis. These methods are preferably used when the peptide is relatively short (i.e., 10 kDa) and/or when it cannot be produced by recombinant techniques (i.e., not encoded by a nucleic acid sequence) and therefore involve different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the polypeptide of the present invention are desired, the polypeptide can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680, Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463. Combinatorial chemical, antibody or peptide libraries may be used to screen a plurality of peptides.

It will be appreciated that the polypeptide core of the present invention can be artificially modified in order to include the desired properties.

Modification of polypeptides (e.g. polypeptides with a catalytic activity such as enzymes) can be effected using numerous protein directed evolution technologies known in the art [for review see Kuchner and Arnold (1997) TIBTECH 15:523-530].

Typically, directed enzyme evolution begins with the creation of a library of mutated genes. Gene products that show improvement with respect to the desired property or set of properties are identified by selection or screening, and the gene(s) encoding those enzymes are subjected to further cycles of mutation and screening in-order to accumulate beneficial mutations. This evolution can involve few or many generations, depending on the progress observed in each generation. Preferably, for successful directed evolution a number of requirements are met; the functional expression of the enzyme in a suitable microbial host; the availability of a screen (or selection) sensitive to the desired properties; and the identification of a workable evolution strategy.

Examples of mutagenesis methods which can be used in enzyme directed evolution according to this aspect of the present invention include but are not limited to UV irradiation, chemical mutagenesis, poisoned nucleotides, mutator strains [Liao (1986) Proc. Natl. Acad. Sci. U.S.A 83:576-80], error prone PCR [Chen (1993) Proc. Natl. Acad. Sci. U.S.A 90:5618-5622], DNA shuffling [Stemmer (1994) Nature 370:389-91], cassette [Strausberg (1995) Biotechnology 13:669-73], and a combination thereof [Moore (1996) Nat. Biotechnol. 14:458-467; Moore (1997) J. Mol. Biol. 272:336-347].

Screening and selection methods are well known in the art [for review see Zhao and Arnold (1997) Curr. Opin. Struct. Biol. 7:480-485; Hilvert and Kast (1997) Curr. Opin. Struct. Biol. 7:470-479]. Typically, selections are attractive for searching larger libraries of variants, but are difficult to device for enzymes that are not critical to the survival of the host organism. Further more, organisms may evade imposed selective pressure by unexpected mechanisms. Less stringent functional complementation can be useful in identifying variants which retain biological activity in libraries generated using relatively high mutagenic rates [Suzuki (1996) Mol. Diversity 2:111-118; Shafikhani (1997) Biol. Techniques 23:304-310; Zhao and Arnold (1997) Curr. Opin. Struct. Biol. 7:480-485].

According to this aspect of the present invention hybridization (i.e., formation of double stranded nucleic acid sequences by way of complementarity) of at least two input nucleic acid sequences with two complementary nucleic acid sequences modifies the at least one output nucleic acid sequence and optionally the polypeptide core.

As used herein the phrase “modifies” refers to any structural (conformational) or chemical modification, in a direct or indirect manner, of the output nucleic acid sequence and optionally of the polypeptide core as is further described hereinbelow. Modification of the output nucleic acid sequence can be for example addition, removal, substitution or denaturation of nucleic acid(s). Preferably, the hybridization of the two input nucleic acid sequences elicits (via formation of a polypeptide complex as is further described hereinbelow) output nucleic acid sequence denaturation (see for example, FIGS. 3d-e).

As is mentioned hereinabove, the molecular entity of the present invention is capable of forming a logic gate. Examples of such logic gates include the AND gate, the OR gate, the NOT gate, the NOR gate, the XOR gate, the NAND gate and XNOR. It will be appreciated that for the formation of a logic gate three distinct molecular entities can be utilized, each with a distinct combination of input and output nucleic acid sequences, yet, the output nucleic acid sequences of two molecular entities are selected complementary to the two input nucleic acid sequences of the third molecular entity.

Preferably, at least one of the two catalytic functions of the polypeptide core is a kinase activity. The kinase used in the polypeptide core of the present invention can be any kinase known in the art. It will be appreciated that some kinases are positively regulated (i.e., capable of phosphorylating a substrate when being phosphorylated themselves on specific amino acid residues) or negatively regulated (i.e., capable of phosphorylating a substrate only when being not phsophorylated on specific amino acid residues). It will be appreciated that the type of kinase used in the polypeptide core of the present invention depends on the type of logic gate the molecular entity of the present invention is design to execute. For example, as is described in Example 4 of the Examples section which follows, for the AND gate a positively regulated kinase can be used. Alternatively, for the NAND gate, a negatively regulated kinase is preferred.

As is further described in Example 4 of the Examples section which follows, suitable negatively regulated kinases include the DRP-1 of the DAP-kinase family of Ca²⁺/calmodulin (CaM)-regulated Ser/Thr kinases (25, 26). DRP-1 was cloned from various species including homo sapiens [mRNA—GenBank Accession No. AF052941, protein—GenBank Accession No. AAC35001.1], murine [mRNA—GenBank Accession No. AF052942, protein—GenBank Accession No. AAC35002.1; Boaz Inbal, et al., 2000, Mol. Cell. Biol. 20(3): 1044-1054] and C. elegans [GenBank Accession No. NP_{—}741403].

Preferably, the polypeptide core of the molecular entity of the present invention includes at least a functional portion of the DRP-1 amino acid sequence as set forth in GenBank Accession No. AAC35001.1. As used herein the phrase “functional portion” refers to a portion of the polypeptide which is required for forming a homodimer and which is capable of phosphorylating a substrate in a negatively regulated manner, e.g., when being unphosphorylated on the serine residue at position 308 (Shani G, et al., 2001).

As described hereinabove, the hybridization of at least two input nucleic acid sequences with two complementary nucleic acid sequences may optionally modify the polypeptide core. Such modification can be any protein modification known in the art including, but not limiting to phosphorylation, dephosphorylation, methylation, demethylation, acetylation, deacetylation, glycosylation, acylation, amidation, imitation, sulfation, and the enzymes that remove such modifications. Preferably, the modification of the polypeptide core is phosphorylation.

According to one preferred embodiment of the present invention the polypeptide core comprises a phosphatase activity which, as explained in Example 4 of the Examples section which follows, can be used to form the NOT gate.

Preferably, the present invention contemplates a composition-of-matter which comprises a plurality of the molecular entities of the present invention. Thus, an input nucleic acid sequence of one molecule can hybridize to an output nucleic acid sequence of another molecule and thus elicit the modification of an output nucleic acid sequence and optionally also of the polypeptide core.

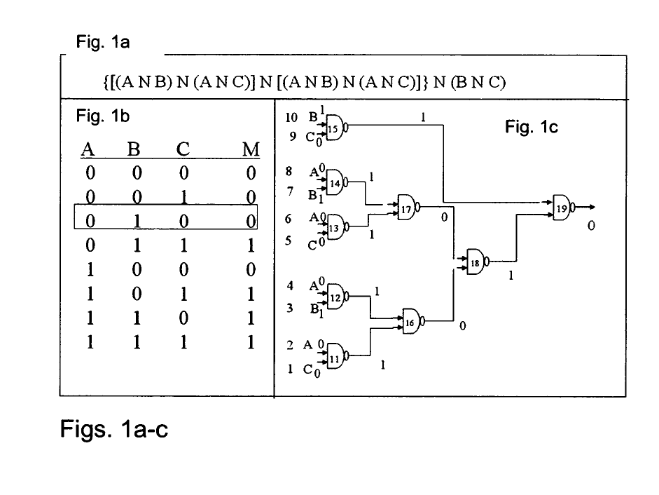

As is mentioned hereinabove, for the formation of a logic gate a combination of three of the molecular entities is utilized (see a schematic illustration of such combination in FIG. 3b). However, it will be appreciated that in order to execute a computational task using the molecular entities of the present invention, the composition-of-matter preferably includes molecular entities capable of forming at least two layers of logic gates (for a schematic illustration of such layers see FIG. 1c). It will be appreciated that for a computational task which involves various detectable markers (e.g., mRNA to various genes, monitoring the level of glucose, urea and salts), multiple layers of logic gates are required. The present invention therefore contemplates composition-of-matter capable of forming multiple layers of logic gates using various combination of input or output tags using the basic molecular entity described hereinabove.

Preferably, the composition-of-matter of the present invention further comprising ATP which is utilized as an energy source. It will be appreciated that ATP can be also provided from the biological environment in which the composition-of-matter is present.

In addition, the composition-of-matter may further include salts and/or buffers capable of stabilizing the molecular entities comprised in the composition. It will be appreciated that the agents comprising the composition-of-matter of the present invention (e.g., the nucleic acid sequences, polypeptides, salts) are preferably prepared in a highly pure form using common purification techniques used for pharmaceutical and/or diagnostic agents (e.g., according to FDA approved techniques).

As described in the Examples section which follows, the computation takes place in a solution containing all the required molecules, which are allowed to diffuse freely. In the solution, the basic logical element is a set of two input molecules (i.e., molecular entities with active output nucleic acid sequences) and one output molecule (i.e., a molecular entity with active input nucleic acid sequence), as described hereinabove and in the Examples section which follows.

Each set is preferably configured to allow the computation of a universal gate such as NAND or NOR. The basic logical elements assemble a set of tag nucleic acid sequences that functions as a biological logic circuit, which is configured to perform a certain logical computation, as described in the Examples section. In order to assemble the biological logic circuit, the basic logical elements are divided to different layers, as exemplified in Example 2 of the Examples section which follows.

In one embodiment of the present invention, the basic logical elements that assemble the biological logic circuit are placed in a closed container (e.g., a device). The container is preferably comprised of a semi-permeable membrane walls. The walls are used for blocking the molecular entities of the biological logic circuit within the container while allowing certain molecules or ions to pass therethrough by diffusion. Particles are separated based on their molecular size and shape, with the use of pressure. Such a membrane can be implemented since the molecules of basic logical elements are attached to proteins (i.e., the polypeptide cores) which have relatively large spatial area in relation to other molecules. A semi-permeable membrane such as cellulose acetate, polyvinyl acetate, ethylene vinyl acetate or the like may be used. Preferably, the semi-permeable membrane walls allow the diffusion of molecules of different chemicals, toxins, minerals, antagonists, mRNA, DNA, and any other molecule is designed to be assessed by the biological logic circuit.

Such containers may be used for assessing the level of predefined substances in a certain solution, in vitro, or in as a medical device, in vivo.

The molecules, which are placed in the container, are configured for carrying out a computation that follows the logic of a Boolean circuit. This ability may be used for assessing biological reactions. Preferably, the input molecules in the basic logical elements of the first layer of the biological logic circuit are configured to sense biological markers such as chemicals, minerals, nutrients (e.g., glucose) or several disease markers (e.g., overexpression or underexpression of certain genes).

For example, overexpression of a certain gene (e.g., excess of a specific mRNA) can be used to activate the input or output nucleic acid sequence of the first layer of input molecules by way of hybridization, essentially as described in Example 2 of the Examples section which follows.

Alternatively, sensing biological markers such as chemicals, minerals, nutrients (e.g., glucose) can be performed by attaching molecules which are sensitive to excess of such markers in a way which triggers a conformational change in such molecules. For example, the molecular entity of the present invention which is used as a first layer input molecule can be bound to a glycoprotein in such a way that the output nucleic acid sequence not being exposed (i.e., inactive). Upon an increase in the glucose level, the glycoprotein can be subjected to additional glycosylation which induces a conformational change in the glycorprotein-molecular entity complex, thus resulting in exposure of the output nucleic acid sequence (i.e., activation).

Additionally or alternatively, the molecular entity of the present invention can be conjugated to an antibody which is capable of specifically binding a glycosylated polypeptide such as hemoglobin (e.g., glycosylated hemoglobin A1C which is for monitoring diabetes) and which upon interaction with such molecule induces a conformational change in the molecular entity of the present invention, thus resulting in exposure of the output nucleic acid sequence and activation of the logic circuit.

Regardless of the trigger used to initiate the process (i.e., activates the first layer input molecules), once the input molecules are activated the computation process begins. The base pairing (hybridization) initiates the computation process which is defined by the biological logic circuit. In the end of the computation process, a number of output molecules are released to the inner space of the container. The released output molecules represent the outcome of the computation process. Preferably, the semi-permeable membrane walls are adapted to allow the diffusion of the output molecules which are released in the end of the computation process.

In one embodiment of the present invention, a digital imaging system for quantifying the released output molecules is used. The digital imaging system is used for converting the released output molecules to a digital signal. The digital signal represents the outcome of the computation process and may be used as a control signal for controlling different medical devices or laboratory tools.

Preferably, the released output molecules are designed to be base-paired or bind with fluorescent reporter molecules. After the foal phase of the computation is determined, the output molecules diffuse and find target fluorescent reporter molecules.

The binding of the output molecules with the target fluorescent reporter molecules, preferably changes the fluorescence level of the fluorescent reporter molecules. As commonly known, the fluorescence of fluorescent reporter molecules can be recorded using digital imaging system. Such a system usually comprises image sensors or fluorometers which are used for capturing an image that reflects the fluorescence of the fluorescent reporter molecules. Preferably, one or more illumination modules are used for illuminating a predefined area. The illumination intensifies the fluorescence of the fluorescent reporter molecules.

When fluorometers are used, the digital imaging system is directed to a digital frequency domain for measuring the fluorescence response of the fluorescent reporter molecules when excited by the illumination module. Preferably, the fluorometer output is acquired and analyzed. The acquisition involves exciting a sample, thereby causing it to emit fluorescent light. In one embodiment, the emitted fluorescent light is captured and down-converted to a more manageable frequency using the fluorescent reporter molecules and reference photomultiplier tubes (PMT) that mix a cross-correlation frequency. The correlation signal from the PMT is now an electric signal as opposed to a light signal. The phase and modulation information from the response of the sample is carried by a discrete waveform at the correlation frequency. This information is processed by a processing unit.

In another embodiment, a diode, charge-coupled device (CCD) array, or a Complementary Metal Oxide Semiconductor (CMOS) array is coupled with a software module which is used for analyzing the spectral and frequency response of the fluorescent reporter molecules at discrete x-y locations of a certain captured area. The software module is used for quantifying the captured fluorescence, and sending the results to a computer screen, a data file or a designated electrical circuitry. The quantifying is preferably done using image processing techniques. Such image processing techniques are generally well known in the art and are, therefore, not described here in greater detail.

The output of the digital imaging system may be used for controlling devices for administrating Electro-muscle stimulation (EMS). EMS, as commonly known, is a technology that utilizes a conductive pad or electrode to externally apply a very weak current to a muscle or group of muscles and thereby cause them to contract. The electrode receives an electric stimulation signal from an external voltage/current source, such as an EMS machine. The stimulation signal can be adjusted in amplitude, polarity, frequency, waveform, etc. Such an embodiment may allow the converting of biological reactions to stimulation which are used for operating a muscle or group muscles.

The output of the digital imaging system may be used for administrating the release of different substance such as hormones, or any other therapeutic agent to the blood. Preferably, the container is coupled to an administration device that comprises therapeutic agents such as insulin, chemotherapy agents, various receptor antagonists, receptor agonists and the like. The container is adapted to control the administration of the therapeutic agents from the administration device, according to the output of the computation process.

In another embodiment of the present invention, the aforementioned solution is used as a biologic computational unit. The solution that functions as a biological logic circuit may be used as a computing unit. Preferably, molecules, which are adapted to be base-paired with the two input molecules of the basic elements of the first layer, are added to the solution. The added molecules initiate the computing process, as described in the examples section which follows. Preferably, added molecules are chosen to reflect certain data which has to be compute.

Since the solution is not limited to a certain amount of basic logical elements, the biological logic circuit, which is represented in the solution, may be designed to compute in parallel large amount of data that is represented by the added molecules.

The outcome of the computation is represented by the molecules which are released in the end of the computation process. This outcome may be analyzed and transferred to digital representation, as described above.

Thus, the device of the present invention which comprises the composition-of-matter described hereinabove can be used for extra-corporeal or intra-corporeal clinical applications. The advantages of using such a biological computing unit instead of a “common” computing unit include the low energy consumption [hydrolysis of several ATP molecules per basic logical operation (about 10⁻¹⁹ joule), as compared with more than 10⁻⁹ joule per operation for current supercomputers (1)], the ability to use endogenous ATP that is present in the body, and its relatively low size. In addition, since the device comprises molecular entities made of polypeptides and nucleic acids, it is biocompatible and non-toxic to the subject.

As described hereinabove, the biological computing unit of the present invention can be used to monitor the level of various markers such chemicals, minerals, nutrients (e.g., glucose) or several disease markers (e.g., overexpression or underexpression of certain genes). For any specific task, a specific combination of the molecular entities of the present invention which form the universal gates (e.g., NAND) can be used. The outcome of the computation process can result, for example, in a release of a therapeutic agent (as described hereinabove) and/or the blockage of a specific physiological process (using e.g., an antagonist molecule or by way of hybridization to target endogenous sequences).

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### The Molecular Computing Unit: Choice of Gates for the Computation Scheme

The following example schematically describes in abstract terms, the design of a bio-molecular system that is capable of carrying out a computation that follows the logic of a Boolean circuit based on NAND gates.

The choice of universal Boolean gates—Boolean algebra deals with calculating truth values (TRUE or FALSE) of logical statements and is the underlying mathematical tool of any digital circuit. Every Boolean function can be expressed using the two gates of AND and NOT (or OR and NOT). However, to keep the design simple and uniform, a single universal gate that can be combined to express any function was chosen. The NAND gate is one such gate (NOR is another possible gate). The NAND gate is an AND gate with the output INVERTED. The AND gate outputs “1” when ALL of the inputs are “1”, otherwise the output is “0”. The INVERTED gate (or the NOT gate) performs the inversion of the input. When the input is “1”, the output is “0”; when the input is a “0”, the output is a “1”. A NAND gate outputs “1” unless its two inputs are “1”, in which case its output is “0”. NAND is universal since it can express the standard gates, OR, AND and NOT:

NOT A≡(A NAND A)

A AND B≡(A NAND B) NAND (A NAND B)

A OR B≡(A NAND A) NAND (B NAND B)

The NAND can also be used for implementing NOR:

A NOR B≡((A NAND A) NAND (B NAND B)) NAND ((A NAND A) NAND (B NAND B))

An example of logical network based on NAND gates is shown in FIGS. 1a-c. This circuit is further discussed in more details hereinbelow, but its general features are common to all logical circuits. Note that each gate has two input and one output ports, gates are wired in layers in such a way that the output of one gate is the input to the next gate, and the computation propagates from the input layer to the output element according to Boolean arithmetic, as implemented by the gates.

Simplicity: The choice of single species molecules to carry out the logic gates—A single species of molecule can carry out the NAND gate logic, and thereby forms the basis of the computation. To achieve this goal, the molecule must perform three tasks. The first is recognition, i.e., only the appropriate molecules may recognize and interact with each other. For this purpose, the design requires that each molecule have three recognition sites, two to recognize incoming molecules (i.e., input sites) and one to recognize the target molecule (i.e., an output site). The second task is synchronization: i.e., interactions must occur only between active molecules, those that are at the appropriate stage of the computation. To enable synchronization, the design requires that the recognition sites are initially blocked or inactive, and become active at a desired time during the computation. The third task is the actual computation, i.e., the change in the state of the molecules such that they will carry the correct logical value. As an example, a two state mechanism that provides reversible modification of the molecule, such that one state represents ‘zero’ and the other state represents ‘one’ can be used.

Molecules function as input ports, output ports and wires—The basic element is therefore a molecule that includes two catalytic domains, performing the tasks of activation and computation, and three recognition tags to enable recognition of the molecule by the specific computational elements with which it must interact in the logical circuit. The tags are encoded such that an output tag of a given element recognizes the input tag of its designed target. Thus, a pair of complementary tags provides a ‘wire’ connecting the output of one gate with the input of another. All molecules in the network have the same catalytic domains, but have different tag sequences that uniquely define their input and output interactions. Two tags are used to define input interactions and one is used to define an output interaction. The input tags are always active and ready to receive a signal. The output tag is initially blocked. This block is removed once the molecule acquires its logical value (either zero or one). The computation takes place when two active input molecules bind the element, and is depended on the logical state of the input molecules. Following the logic of e.g., a NAND gate, the output molecule obtains the value of “zero” only when both input molecules are “one”; in all other cases the output molecule will assume the value of “one”.

Molecules with complementary tags associate to form complexes which further transfer information—Computation takes place in a solution containing all the required molecules, which are allowed to diffuse freely. Molecules collide randomly, but only molecules that have complementary tags associate to form complexes which can transfer information. At the start of the computation, only the molecules that represent input to the system, i.e., the first layer in the circuit, have an accessible output tag. Thus, only these molecules can interact effectively with their targets, while the output tags of all other molecules are inaccessible, preventing them from interacting with additional elements before they receive a valid input signal. However, their input tags are accessible, making them available to receive signals from molecules that have already been activated. In subsequent phases, only molecules that have acquired an accessible output tag can further interact.

Mechanism of computation via formation of three-molecules complexes—Since each molecule has two accessible input tags, three molecule complexes are formed. Within each complex, the computation and the synchronization steps take place. The computation will set the logical state of the output molecule depending on the logical state of the two input molecules according to the logic of a NAND gate, and synchronization is provided through activation of the output molecule by making its output tag accessible. Over time the output molecule diffuses and finds its target molecule, allowing the process to continue until the logical state of the molecules in the final phase of the computation is determined.

As mentioned above, each basic element is characterized by the specific combination of its input and out tags. Multiple identical copies (on the order of 10⁹, see below) of each element are present in the system. Thus, for example, as is shown in FIGS. 1a-c, if element number 15 is required to interact with element number 19, any one of the active copies of element number 15 can interact with any one of the copies of elements 19, based on their complementary tags. As is further discussed below, this parallelism can be used to facilitate error detection and correction.

### Example 2

### The Biological Implementation of the Molecular Computing Unit

The following example describes a detailed account of how the biological reactions chosen can be used to implement the scheme. The following reactions are certainly not the only possibilities for implementing a general design of a computational network, and some possible alternative mechanisms are also suggested. Regardless of the actual reactions that are ultimately used in engineering a practical implementation, the model proposed here is a general one, in the sense that the same design can be used to perform any logical calculation via biological computation.

The model proposed above is based on general ideas but must be implemented using specific biological processes and reactions. In this section, one set of reactions is suggested that could carry out the tasks required. The feasibility of these reactions is further described hereinbelow.

Formation of “wires” is based on recognition between DNA sequences—Recognition can be achieved through hybridization of complementary DNA tags. Each tag is composed of a single stranded DNA oligomer that is covalently attached to the protein part of the molecules. Binding of complementary tags provide a localization effect, effectively enabling the logical computation for a single gate. In a sense, these tags are used as “wires” in this diffusive network. To achieve synchronization, the output DNA tags can be blocked by a complementary DNA strand (thus being in a double-stranded form), which is removed only after the associated gate is formed. The removal is achieved by activation of an appropriate exonuclease that digests the blocking strand, thereby exposing the output tag (making it single-stranded) and renders it active.

Computation can be achieved using phosphorylation reactions—To achieve the computation a phosphorylation domain can be included that is capable of performing a conditional phosphorylation reaction, such that the logical state of the output molecule (i.e., whether phosphorylated or not) is dependent on the phosphorylation state of the two input molecules. The phosphorylation state of the input molecules is configured to reflect the desired input logic. FIG. 2 depicts a schematic view of the basic computational element.

Computation is depended on the phosphorylation state of the two input molecules—Computation takes place in a solution containing a mixture of all the molecules, which are allowed to diffuse freely. Molecules collide randomly, but only molecules that have complementary DNA tags associate by hybridization to form complexes with significant half-lives. The input molecules, i.e., the first “layer” of the circuit can be set using several configurations. One option is that their output DNA tags are exposed, rendering them “active” and their input tags are “covered” (e.g., using a complementary oligonucleotide) preventing them from interacting with other target molecules. Alternatively, the input molecules can be designed such that their input tags are covered (i.e., inactive) and their output tag is also covered. Thus removal of the cover (i.e., an hybridizable polynucleotide which forms double stranded nuclei acid) from the output DNA or the input tag (i.e., activation of the input molecule) can be conditional and depended on the “biological environment”. For example, removal of the cover can be effected by a competitive hybridization with another polynucleotide sequence (DNA or RNA) that is present in the biological environment of the molecular computing unit (e.g., blood). For example, excess of mRNA molecules can trigger the hybridization between the “cover DNA” (i.e., the hybridizable polynucleotide which forms double stranded nucleic acid) and the mRNA and thus expose the output or the input tags, rendering the input molecule active. Alternatively, the input tag can be activated by a change in a chemical, mineral or nutrient (e.g., glucose) via an interphase device sensitive to that change.

The phosphorylation state of the molecules reflects their logic value; For example, phosphorylated molecules can be considered with the logical value of “1”, and de-phosphorylated molecules with the logical value of “0”. All other molecules have their output tags inactive, preventing them from interacting with additional elements before they receive the proper input signal, and their input tags exposed making them available to receive signals from active molecules. The phosphorylation state of the non-input elements is set initially to be de-phosphorylated, i.e., logical value “0”. In subsequent “phases” only molecules that have acquired an activated output tag (e.g. with the blocking oligomer removed in previous “phases”), can further interact.

The biological “gate” is composed of a three-molecule complex: two input molecules and one output molecule—It should be noted that the biological “gate” is somewhat different from an electronic gate. An electronic gate is a single element that receives two input signals, calculates the appropriate logical function and produces an output signal. The biological “gate” is actually a complex that includes three molecules, two input molecules and one output molecule.

Complex formation—FIGS. 3a-f schematically illustrate the interactions leading to complex formation and execution of the NAND logic gate. Each complex forms in two stages. First one input molecule hybridizes to the first input tag of the target element to form an inactive complex. Upon binding of the second input molecule to the second input tag, the computational complex becomes active and performs the following reactions: The two input molecules interact to form an active dimer that phosphorylates the target molecule if so required by the NAND logic. Thus, the target (output) element is phosphorylated unless both its input molecules are phosphorylated. In addition, an exo-nuclease reaction is activated as a result of association of the two input molecules, digesting the cover of the output tag and leaving the tag exposed, thus making the output tag available for hybridization with the input tag of the next element in the circuit. After some time, the computational complex dissociates, and the activated output molecule seeks its own target, encoded by the complementarity of its output tag with the sequence of an input tag of another molecule. It will be appreciated that the dissociation of the complex is not required in order to proceed with the formation of a subsequent complex. The process can continue, through successive layers of gates, until the final output gate molecules are processed. The result of the computation may be then read from the phosphorylation state of the output molecules.

Multiple copies of the computational elements enable high degree of robustness in the computation—There are many identical copies of each computational element, i.e., molecules that have the same combination of input and output tags. These molecules are interchangeable and each copy can interact with any copy of its designated target molecule. Thus, parallel computation of the same circuit is carried out by a large number of molecules. This redundancy can be utilized as described below to achieve a high degree of robustness in the computation.

### Example 3

### The Majority Circuit

The following example provides a simple biological implementation of a circuit that calculates the majority function of its three inputs.

FIG. 1b provides the Truth Table and the logical design for this circuit. The output is “1” if at least two of its three inputs are “1”, otherwise it is “0”. While this is a very simple calculation that can be performed by many analog processes, the same approach can be used to implement any logical circuit, regardless of its complexity.

The circuit presented in FIGS. 1a-c has 19 elements. Elements 1 to 10 are inputs consisting of molecules similar to the rest of the computational elements, the only difference being that they have preset phosphorylation states, reflecting the required logical values. Gate 19 is the output gate. Thus, the system consists of 19 elements, each with the same protein component, capable of performing the phosphorylation and the exonuclease reactions, but each carrying different DNA tags. There are many copies of each of these elements present in the system.

For the boxed instance in FIG. 1b, variables A and C have the value of “0”, and B has the value “1”. Hence, the computation is initialed by setting input elements 2, 4, 6, 8 corresponding to A, and 1, 5, 9 corresponding to C to a de-phosphorylated state while elements corresponding to B (3, 7, 10) are phosphorylated. All non-input elements are de-phosphorylated. The input elements (1 to 10) have active output tags. All the other elements have their output tags blocked and their input tags active. Wiring is achieved by providing appropriate pairs of complementary tags. For example, the output tag of element 1 is complementary to one of the input tags of element 11, and the output tag of element 11 is complementary to the input tag of element 16, and so on. In the first computation “layer”, no element has its two input elements in state “1” (i.e., phosphorylated), so that phosphorylation reactions take place in all elements. In the second “layer” the inputs to element 16, provided by the output of elements 11 and 12, are both “1”. Similarly, the inputs to element 17, provided by the outputs to elements 13 and 14, are both “1”. Thus, elements 16 and 17 will not be phosphorylated. The computation propagates in this way until the final output element (number 19) forms a complex with elements 15 and 18. Activation of the output tag of element 19 signifies completion of the computation, and the result may be read off by, for example, examination of a fluorescent probe attached to that tag.

### Example 4

### Feasibility of the Molecular Computing Unit

Volume and concentrations—In order to minimize the formation of incorrect interactions the system is established at the most dilute concentration possible. For example, such a system can have a volume of 1 ml. As is further discussed hereinbelow, the system utilizes redundancy in terms of multiple copies of each element to allow for error correction. To achieve this purpose, about 10⁷ copies of each molecule can be used. Assuming a system size of the order of 1000 gates, this will amount to 10¹⁰ molecules in a volume of 1 ml, which is a concentration in the order of 0.1 nM, a suitably dilute system.

Recognition tags—The design calls for the use of single stranded DNA (ssDNA) tags to produce high specificity associations between the appropriate gate elements, i.e., the DNA and protein molecules forming the gates. Thus, the ssDNA tags are used to associate pairs of proteins, the function of which is depended on complex formation (formation of dimer). The function of formed dimer is to enable computation (e.g., by phosphorylation) and further activation (e.g., by exonuclease activity) of the logic gate. The necessary chemistry for fusing protein and DNA is established, for example by covalent attachment of the DNA strand to cysteine residues (18, 19). The association constants between complementary DNA strands are also well understood and predictable (20), forming the basis of temperature dependent melting, as used in PCR and cloning reactions. Synthesis of DNA oligomers is a routine process, partly because of these applications. For example, system tags of about twenty base pairs having binding constants in the 0.1 pM range, would ensure almost complete complex formation at the 0.1 nM concentration of gate elements proposed. As is further discussed hereinbelow, selectivity of tag binding is achieved by ensuring at least eight mismatches between any pair of non-coupled tags.

An alternative is to use PNAs (peptide nucleic acids) which have a peptide like backbone with side chains mimicking DNA bases. PNA is recognized by DNA binding proteins and as a single strand can hybridize to complementary DNA or PNA molecules with high specificity (21, 22). PNA tags can be attached via a peptide bond to the termini of proteins, as well as via a cystine side chain (22). An advantage compared with DNA is that PNA is not digested by nucleases. Note that DNA would still be used to make the covers that initially block the output tags, so that the exo-nuclease will be able to digest them at the appropriate time.

Each gate element carries three tags. These tags can attached to three different sites, as schematically shown in FIG. 2, or more conveniently, fused together with short linkers and attached to one terminus of the protein as shown in FIG. 4. An advantage of this arrangement is that it permits easy automated translation of any logical circuit into its biological equivalent (see below).

Activation of output tags—The output DNA tag of each gate must be blocked from premature association with the element to which it is wired until the logical operation of the gate is complete. The proposed mechanism for ensuring this is to block the tag with a complementary oligonucleotide until activation is required. Activation can be carried out using an exonuclease, which digests the complementary blocking strand. Correct timing is achieved by employing a nuclease that is functional only as a dimer. The dimer interface must be engineered so that significant dimer formation only occurs when the gate complex has been formed. Several commercially available dimeric or tetrameric exonucleases can be used, for example, the Lambda exo and exo III (23), that is capable of digesting double strand DNA, leaving a single intact strand. In fact, a similar idea is used in a product called TaqMan™ that is designed for quantitative PCR measurements in which an oligonucleotide is digested by a DNA polymerase (24).

Gate logic—Logical states are represented by the phosphorylation state of the protein components. There are two possible conventions: phosphorylation represents ‘zero’, or phosphorylation represents ‘one’. NAND logic can be implemented for the former convention by employing a phosphatase activity (removal of a phosphate from the output element if both input elements are phosphorylated) or a kinase for the latter convention (addition of a phosphate to the output element unless both input elements are phosphorylated). In biology, control mechanisms seem to rely much more frequently on kinases than on phosphatases, and so there is a much richer choice of possible kinase enzymes to employ. In case a kinase activity underlies the computation process, the kinase system includes the following properties: First, the enzyme must be active only as a dimer. Second, the dimeric form of the enzyme must be able to phosphorylate other monomers. That is, the dimer of molecules should add a phosphate to a monomeric form of the same molecule. Third, it exerts negative control, i.e., it is inactive only when both subunits are phosphorylated. It will be appreciated that if other logical gates are used in the construction of the molecular computing unit, then the enzymes (e.g., phosphatases, kinases, methylases, acetylases) used to execute the logic gate are selected to perform other combinations of activities.

Since many kinases are active as dimers, and many are auto-catalytic, the first two requirements are relatively easy to achieve. For the third requirement, negative control of kinases is needed (i.e., kinases that work only when at least one monomer is not phosphorylated). Although it is much more common for kinases to be activated by phosphorylation (i.e., positively regulated kinases), there are kinases which are negatively regulated and thus can be used for the computational molecule. A non-limiting example of such a kinase is the DRP-1 of the DAP-kinase family of Ca²⁺/calmodulin (CaM)-regulated Ser/Thr kinases (25, 26). These molecules function as positive mediators of programmed cell death. The protein combines two of the desired properties—it is active only as a dimer, and it is most active when un-phosphorylated. When the two subunits are phosphorylated (on Ser308) there is a very significant reduction of its phosphorylation activity. While Ser308 is auto-phosphorylated by the enzyme, its primary phosphorylation target is another protein, a myosin light chain (MLC). Additional control of its activity is provided by calcium dependent calmodulin binding. Thus, a suitable kinase can be formed using protein engineering techniques, starting from DRP-1, or from other proteins.

The proposed biological computing unit presented here concentrates on using NAND gates because NAND is a universal gate that can be used as a single type of gate needed to implement any logical computation. It might turned out that is simpler, protein engineering wise, to design two different biological molecules, one that emulates for example the function of a NOT gate and one that emulates the function of an AND gate. NOT and AND gates, taken together, allow universal computation. Such a pair of gates would require a different cascade of signaling events than the one described here. For example, while for the AND gate a dimeric kinase that is positively regulated (i.e., phosphorylates a target substrate only when its two monomers are phosphorylated) can be used, for the NOT gate an enzyme such as a phosphatase that removes phosphate when phosphorylated combined with a kinase that adds phosphate when non-phosphorylated can be engineered.

Activation by localization—A key feature of the design is high effective local concentration of molecules as a result of the complementarity of the tags. The enzymatic reaction is tuned such that the tag tethered molecules are highly active, while freely diffusing ones are essentially inactive. This is achieved by control of dimerization. Binding of tags to the complementary sequences on a target molecule increases the local effective concentration. Assuming a protein diameter of about 50 Å, and the connecting DNA tether to be of about 250 Å (three tags of about 20 bp each and linkers), the two molecules will be contained within a sphere of about 10⁸ Å (3). The effective concentration will then be of the order of 10 μM. This is 100,000 fold higher than that of the free molecules in the solution (0.1 nM). A dimer association constant of 1 μM will therefore ensure almost complete formation of active enzyme for tag-hybridized molecules. At the same time, it would guarantee a very low amount of dimerization for un-tag complemented molecules: At 0.1 nM concentration, only 1 in 10,000 molecules will be in dimeric form at any time at equilibrium. Similar localization principles have been evoked to explain enzyme rate enhancements (27), and form the basis of methods of detecting naturally occurring protein-protein interactions, for example, in yeast two hybrid assays (28).

Timing within a gate—The output tag of a gate must not be activated until its logical operation is complete. That is, the kinase must add a phosphate to the output element, if required, before the exonuclease exposes enough of the output tag for association with the input of the next gate to occur. Indeed, typical turn over rates for kinases are around 100-1000 per second [see for example, (29)] while an exonuclease would cleave a mask of 20 bp in about 1 to 5 seconds (30).

Speed of computation—How rapidly can these circuits carry out a computation? As mentioned above, digestion of the mask can be completed within 1 to 5 seconds. With 10⁷ copies of each element in a volume of 1 ml, collision rates are significantly faster than that. The phosphorylation rate is also significantly faster. Thus, the exonuclease step is rate limiting. So, a system of a thousand gates, which would have about 10 layers, is expected to complete computation in less than a minute.

Initialization and resetting of the system—For a prototype system, the following initialization and resetting steps are proposed:

A solution of identical untagged and unphosphorylated monomeric molecules is prepared at the required concentration for computation. An aliquot containing sufficient molecules for the input layer is removed. The necessary tags are synthesized in two batches—one containing tags of the input layer (e.g., where the input tags have to be blocked and output tags exposed), the other containing all other tags (where the input tags should be exposed and output tags blocked). The appropriate tags are blocked to prevent pre-mature hybridization between tags. The tags, already appropriately blocked, are introduced into the solutions under ligating conditions and are attached to the protein molecules.

Input elements to be initialized to the logical state ‘one’ are identified by means of their common input tag sequence. A convenient mechanism would be to immobilize the appropriate set of complementary tags on a bead. The bead is then used to extract the corresponding elements from the solution of input layer elements, and to introduce them into a solution containing activated kinase molecules. Following phosphorylation, the elements are released back into the input layer solution by elevating the temperature to melt the tag complexes, (as in a PCR reaction). Similar immobilization methods have been developed for DNA micro-array preparation. This procedure facilitates resetting of the input layer for subsequent computations with different input values.

Computation begins by adding the input layer solution to the main solution. The unmasked output tags on the input elements permit formation of the first layer of complexes. Thereafter, the computation will run to completion automatically.

Activated output molecules can be isolated using the appropriate complementary tags mounted on a bead. Mass spectroscopy then provides a convenient means of determining their phosphorylation states.

For each logical formula, a new combination of tags needs to be assembled. This is not needed for another computation of the same formula with different input values. A computing solution can be prepared for another round of computation by resetting all elements to the unphosphorylated state (using a phosphatase immobilized on a bead), introducing a new set of masking tags, and setting input element phosphorylation states as required.

Possible sources of error—One of the most obvious problems to be addressed in considering computation using biological reactions is that of error in the process. While the selected reactions are of inherent high fidelity, biological processes are never error proof, and reactions might occur between incorrect reactants or produce an incorrect product.

The computational model presented here is sensitive to such problems, since it represents a “tight” computation, in the sense that an error in the outcome of any reaction in the circuit may lead to an incorrect result presented at the output gate.

Several types of errors are possible, and could arise in recognition, synchronization, or computation:

(A) Recognition: Hybridization of unmatched tags. In principle, the specificity of tag recognition can be made as high as desired, by increasing the length of the tags. As noted earlier, quite short tags (approximately 20 bases) are sufficient to achieve an appropriate binding constant. This size will still enable reliable distinction between tags and prevent cross-hybridization. Coding theory [see for example (31)] provides upper and lower limits to the number of code words that differ by a given number of mismatches. For example, for tags of twenty bases, a lower bound on the number of different tags with at least 8 mismatches to any other tag is over 5,000 tags and the upper bound is over 33,000,000 tags. Even the lower bound would be enough for the present prototype system.

(B) Synchronization: Dimerization to form an active complex may occur spontaneously between molecules even without hybridization of tags. As discussed hereinabove, the localization provided by tag binding can be exploited to reduce this to a low level, on the order of 1 in 10,000 complexes in the prototype.

(C) Computation: Kinase action causing phosphorylation inconsistent with the logic of a NAND gate. With an active site split between the components of the active dimer, accidental enzymatic phosphorylation can be reduced to near the spontaneous level observed in the absence of enzyme.

(D) Computation: Failure to phosphorylate when that reaction is the correct logical outcome. The primary cause would be dissociation of one or both of the input molecule tags before the enzymatic reaction takes place. There are then two possible situations. In the first, detachment could occur before full processing of the output tag mask by the exonuclease. In this case, an active complex will eventually reform, and the reaction will again have an opportunity to take place. In the other situation, detachment of an input molecule may take place after full mask processing, but before phosphorylation. In that situation, an error would be propagated. The chances of this occurring can in principle be reduced by decreasing the catalytic rate of the exo-nuclease.

(E) Synchronization: Spontaneous dissociation of the tag masking oligomer not aided by the action of the exonuclease. Since the concentration of tag masks in solution is close to zero, either a very strong interaction and/or a very long half life between the mask and its complementary tag is needed. This is important, since detached tags may associate with output tags on equivalent elements where processing is complete. Spontaneous dissociation can be reduced by making the mask complementarity longer. However, this is probably not necessary since atomic force microscopy data (32) suggest that half-lives of DNA complexes are sufficiently long to minimize tag transfer.

Robustness to errors—It is clear that some errors are unavoidable in such a system, and thus a certain proportion of molecules will carry an incorrect value. On the other hand, in this model, robustness may be achieved by utilizing the fact that the same computation is performed by a large number of molecules. The redundancy of molecules carrying the result provides a mechanism for eliminating errors. For a network with N elements, and an error rate per gate of e, the probability of a correct computation is (1−e)^{N}. If the value of ‘e’ is sufficiently small, a majority vote can be used to obtain a correct result. For example, a system with 100 gates and an error rate of 0.001 per gate would produce highly reliable majority vote results. For cases where the size of ‘e’ is incompatible with the system size, it is not sufficient to take a majority vote on the final results, and the end result of the computation is dependent on obtaining the correct result at each stage. A correction mechanism can be based on the observation that the phosphorylation state of active molecules representing the same gate (i.e., copies of the same gate) should all carry the same value. Different values signify that one of the molecules carries an incorrect result. Since there is no way to know which one carries the correct result, the simple solution would be to eliminate both. The key here is to contain the error in such a way that it will not propagate further along the computation. Such a comparison might be implemented, for example, by a methylation reaction that would be triggered when two active, similarly tagged, molecules with different phosphorylation states interact. Methylation would then block participation of molecules in further interactions.

### Example 5

### Automation and Production

The presently described prototype system of the molecular computing unit of the present invention has a single protein molecular species, containing a kinase and a nuclease domain. A fully developed system may have additional protein components for error control and system resetting. These proteins can be produced using conventional protein expression and purification procedures, and used in the construction of all circuits. The DNA tags are circuit specific, and provide wiring between gates. A circuit is first designed using standard gate notation. Two complementary tag sequences are chosen for each wire connecting the output of one gate to the input of another. The sequences are random, with constraints on composition to ensure appropriate binding constants. Once all tag sequences for a circuit have been generated, an iterative procedure is run, checking to see that no two tags are too similar in sequence, and if they are, generating a new sequence for one of them. Such a library of tags can be pre-prepared and used for all circuits.

The conversion of an electronic circuit to a set of tag oligomer sequences can be fully automatic. The three tag sequences for each gate are combined into one string, with suitable linker regions between them and at the ends. These sequences may be approximately 80 nucleotides long in total, and so can be produced by the same high throughput procedures used in cloning and PCR. Stochiometric amounts of protein and oligomers are then mixed, under conditions that lead to linkage between DNA and protein. In the presently described system there are N*10⁷ protein molecules, and 10⁷ copies of each tag oligomer (where N is the number of gates in the circuit). It is not necessary to have exact numbers. Excess oligomers or proteins are not expected to interfere with the function of the circuit.

Computer simulation—A computer simulation was performed to ensure that the basic design is logically consistent, and evaluated its performance and robustness to errors. Two types of circuit were simulated. One is a generic type whose architecture is of a full binary tree, i.e., a layered structure where each gate is connected to two gates in the previous layer. A system of N levels thus has 2N−1 gates. This allows for simple scalability of the system and simple measurements of the effect of varying parameters. All the input gates were set to the same logical value. The logic of such a network of NAND gates makes the result of the calculation alternate between levels, i.e., if a system of N levels results in “1”, then a system of N+1 levels will results in “0”. The other circuit that was tested was the Majority function described hereinabove. Simulations were done on a two dimensional grid of 600*600 cells in which molecules where allowed to diffuse between cells. Total computation time is defined as the number of steps required for 50% of all the output gates to be activated. Various conditions were investigated.

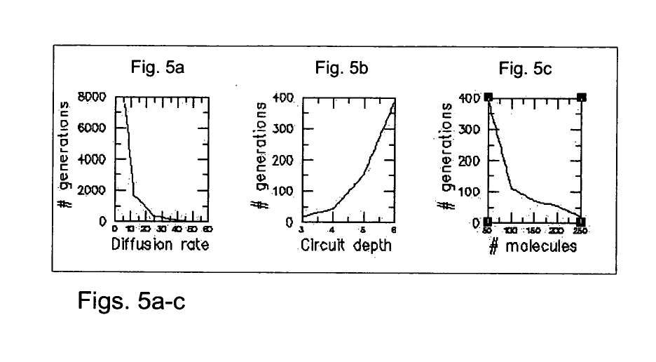

Performance—The effect of diffusion rates was tested in terms of the maximal step size (in grid units) a molecule can take in a single step. Then, the effect of changing the circuit size, and finally the effect of changing the number of copies of each gate that participates in the system were tested. The results for the binary tree are shown in FIGS. 5a-c.

In the first experiment with a binary tree network, the number of copies of each gate was set to 100, and a network with 5 levels (i.e., 31 gates) was used. The diffusion rate (the size of the diffusion step in lattice units) varied from 6 to 60, i.e., for a diffusion rate d, Δx and Δy were changed by a randomly chosen value between 0 and d. As expected, the computation time in terms of the number of generations (each generation is one move of each molecule) decreased significantly as the diffusion rate increased (FIG. 5a). In the next experiment, the diffusion rate was set to 36 lattice points per move, and the size of the circuit was changed. Networks of depth 3 (i.e., binary trees with 3 layers, containing 7 gates) to depth 6 (63 gates) were investigated. The computation time increased exponentially with the depth of the circuit (FIG. 5b), whereas in a silicon-based computation, the time increases approximately linearly with the circuit depth. Next, the time dependence on the level of parallelism in the system was tested in terms of the number of copies of each gate. At a diff usion rate of 36 lattice points per move it can be seen (FIG. 5c) that the performance improves exponentially with the number of copies. This property of the protein based system offsets the exponential time dependence on gate depth. A simple extrapolation suggests that with the intended number of copies (10⁷) circuits of depths of up to 20 could be handled. However, notice that the requirement of the presently described system is that 50% of the output elements will complete their computation before the result is over determined. In practice, with 10⁷ copies of the output elements, even when 1% of the copies (i.e., 10⁵) are completed, the result can be reliably determined. This will enable circuits with significantly greater depths.

Robustness to errors—Next, the performance of the network was tested in the presence of errors. The errors were simulated using a single parameter that specifies the probability that the result of a gate operation is not the correct NAND outcome. The results, for the majority function, with 100 copies and a diffusion rate of 36 (Table 1) show the relationship between error rate and circuit accuracy.

TABLE 1
Relationship between gate error rate and the fraction of output gates
providing the correct logical result

| | |
|---|---|
| | Error detection |
| No error detection | & Elimination |

| % Error rate | % Correct | % Correct | % Yield |
|---|---|---|---|
| 1 | 100 | 100 | 83 |
| 2 | 92 | 98 | 75 |
| 5 | 74 | 97 | 48 |
| 10 | 61 | 92 | 24 |
| 20 | 55 | 100 | 5 |

Table 1: The majority circuit was used, with 100 copies of each gate.
‘% Error rate’ is the probability for error within each gate;
‘% Correct’ is the proportion of cases in which an output gate ended with the correct logical value. Error correction (last two columns) is performed by comparing the logical value of each pair of equivalent processed gates throughout the circuit. Any pair of gates with inconsistent values are removed from further computation.
‘% Yield’ is the fraction of output gates remaining after this elimination process. A majority vote over all output gates will still yield a correct outcome for the entire system down to 10% error rate for individual elements for this small circuit. Error detection and elimination provides a correct output for up to 20% error rate. Beyond that, all gates would be eliminated, and thus there is no outcome.

As is shown in Table 1 hereinabove, up to error rate of 10%, the output accuracy is still reasonable and the correct answer can be obtained by taking the majority result over the set of output gates. If the overall error rate in each elementary calculation is higher, the percentage of the correct answer gets too close to 50% to allow reliable determination of the outcome. Thus, it is necessary to employ an active mechanism of error detection and elimination. A method for error elimination was simulated in which every active molecule undergoes a validation check, by comparing its output value with another active copy of the same molecule, as discussed above. Such a mechanism produces a dramatic improvement in the robustness of the results (Table 1). The success rate of the computation is above 90% up to the highest error rate tested, 20%. This success is achieved at the cost of eliminating some molecules and greater system complexity. With the low number of copies used here, only 24% of molecules in the output layer remain when correcting a 10% error rate, and only 5% remain with an error rate of 20%.

As discussed in Example 4, hereinabove, no single error appears to significantly affect the final outcome (i.e., error rates can be controlled to a very low level), and for small circuits, at least, error correction should not be necessary.

Analysis and Discussion

The design of the molecular computing unit of the present invention presented hereinabove addresses the following questions.

1. How can logical gates (i.e., switching) be implemented by a protein-based system?

Logical operations are performed by phosphorylation reactions that implement the logic of a NAND gate.

2. How can wiring between gates be implemented?

Wiring is implemented by single strand DNA tags that are attached to each protein. A pair of complementary tags wire the output of one gate to the input of another.

3. What are the “tokens” of the computation, i.e., how is the computation carried out from input to output?

The tokens of the computation are defusing molecules with two different possible phosphorylation states. The phosphorylation state of these molecules carries the information transferred from the input to the output of the circuit.

4. Since the biological processes utilized in the system vary in their reaction speed, how can the timing of the computation be synchronized?

Synchronization of the network is achieved by blocking the output tags of each molecule until that molecule has become associated with the appropriate input molecules. Unblocking of tags is performed by an exonuclease which is activated upon complex formation.

5. What are the expected errors in the process and how can these errors be contained?

Problems that might occur have been identified and discussed. Conditions were identified to minimize the possibility of error in each process. Furthermore, the redundancy in the system (e.g., having 10⁷ copies of the circuit) enables reliable computation of the entire system even when the individual reactions might be erroneous. If the error at each stage were to become too large to be contained, then more active error detection mechanisms could be added. Possible approaches to this problem are described.

6. How can the design of such a computation device be automated such that it will possible to take a layout of a regular electronic circuit and produce a biological equivalent?

The process of converting a logical circuit to biological computation can be automated since the design uses a single protein species to build all the logical gates. Wiring between gates is created by synthesizing appropriate DNA tags and attaching them to the protein molecules, using standard technology.

The system is based on two major engineering tasks: attaching DNA (or PNA) tags to proteins and their use to facilitate protein-protein interactions, and engineering a dimeric negatively controlled auto-kinase. Once a working system has been constructed, the same component design can be used for any logical circuit, and thus any technological improvement in the elementary processes will directly benefit every computation.

In selecting the biological mechanism on which to base a computational device, the present inventors have considered the following issues: a system in which the switching is binary, and can toggle between two well defined and well separated states; a system in which the basic element is a single molecule and not itself a network; a system which utilizes proteins that have a natural function close to that required in the computation. Thus, it should be possible to tap into the repertoire of natural reactions in order to find the most suitable starting point for the design; a system in which reactions can be easily chained together to achieve a flow of computation.

It is conceivable that the technology presented here exhibits a large potential, especially in medical applications. For example, the molecular computing unit of the present invention can be designed to monitor various parameters in the body (e.g., in the blood stream) and using the logic gates computation analysis to administer a suitable dose of a drug (e.g., insulin, chemotherapy).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

1. Adelman, L M. Molecular computation of solutions to combinatorial problems. Science 266:1021-1024.

2. Yurke B, Turberfield A J, Mills A P Jr, Simmel F C, Neumann J L. A DNA-fuelled molecular machine made of DNA. Nature 2000; 406:605-608.

3. Braich R S, Chelyapov N, Johnson C, Rothemund P W, Adleman L. Solution of a 20-variable 3-SAT problem on a DNA computer. Science 2002; 296:499-502.

4. Benenson Y, Paz-Elizur T, Adar R, Keinan E, Livneh Z, Shapiro E. Programmable and autonomous computing machine made of biomolecules. Nature. 2001; 414:430-434.

5. Benenson Y, Adar R, Paz-Elizur T, Livneh Z, Shapiro E. DNA molecule provides a computing machine with both data and fuel. Proc Natl Acad Sci USA. 2003; 100:2191-2196.

6. Benenson Y, Gil B, Ben-Dor U, Adar R, Shapiro E. An autonomous molecular computer for logical control of gene expression. Nature 2004;429:423-439.

7. Bode B W, Sabbah H T, Gross T M, Fredrickson L P, Davidson P C. Diabetes management in the new millennium using insulin pump therapy. Diabetes Metab Res Rev Suppl 1 2002:S14-20.

8. Turing, A. M. On computable numbers, with an application to the Entcheidungproblem. Proc. Lond Math. Soc. II Ser. 1936; 42:230-265.

9. Braun E, Eichen Y, Sivan U, Ben-Yoseph G. DNA-templated assembly and electrode attachment of a conducting silver wire. Nature. 1998; 391:775-778.

10. Keren K, Krueger M, Gilad R, Ben-Yoseph G, Sivan U, Braun E. Sequence-specific molecular lithography on single DNA molecules. Science 2002; 297:72-75.

11. Morton-Firth C J, Shimizu T S, Bray D. A free-energy-based stochastic simulation of the Tar receptor complex. J Mol Biol. 1999; 286:1059-1074.

12. Elowitz M B, Leibler S. A synthetic oscillatory network of transcriptional regulators. Nature. 2000; 403:335-338.

13. Gardner T S, Cantor C R, Collins J J. Construction of a genetic toggle switch in Escherichia coli. Nature. 2002; 403:339-342.

14. Chen Z, Govender D, Gross R, Birge R. Advances in protein-based three-dimensional optical memories. Biosystems 1995;35:145-151.

15. Ashkenazi G, Ripoll D R, Lotan N, Scheraga H A. A molecular switch for biochemical logic gates: conformational studies. Biosens Bioelectron 1997; 12:85-95.

16. Sivan S, Lotan N. A biochemical logic gate using an enzyme and its inhibitor. 1. The inhibitor as switching element. Biotechnol Prog 1999; 15:964-970.

17. Bray D. Protein molecules as computational elements in living cells. Nature 1995; 376:307-312.

18. Corey D R, Schultz P G. Generation of a hybrid sequence-specific single-stranded deoxyribonuclease. Science 1987; 238:1401-1403.

19. Bruick R K, Dawson P E, Kent S B, Usman N, Joyce G F. Template-directed ligation of peptides to oligonucleotides. Chem Biol 1996; 3:49-56.

20. SantaLucia J Jr. A unified view of polymer, dumbbell, and oligonucleotide DNA nearest-neighbor thermodynamics. Proc Natl Acad Sci U S A. 1998; 95:1460-1465.

21. Corey D R Peptide nucleic acids: expanding the scope of nucleic acid recognition. Trends Biotechnol 1997; 15:224-229.

22. Zhang X, Ishihara T, Corey D R. Strand invasion by mixed base PNAs and a PNA-peptide chimera. Nucleic Acids Res. 2000; 28:3332-3338.

23. New England Biolabs 2002-2003 catalog pp 107-108.

24. Holland P M, Abramson R D, Watson R, Gelfand D H. Detection of specific polymerase chain reaction product by utilizing the 5′----3′ exonuclease activity of Thermus aquaticus DNA polymerase. Proc Natl Acad Sci USA. 1991; 88:7276-7280.

25. Shani G, Henis-Korenblit S, Jona G, Gileadi O, Eisenstein M, Ziv T, Admon A, Kimchi A. Autophosphorylation restrains the apoptotic activity of DRP-1 kinase by controlling dimerization and calmodulin binding. EMBO J. 2001; 20:1099-1113.

26. Shohat G, Shani G, Eisenstein M, Kimchi A. The DAP-kinase family of proteins: study of a novel group of calcium-regulated death-promoting kinases. Biochim Biophys Acta. 2002; 1600:45-50.

27. Jencks W P. From chemistry to biochemistry to catalysis to movement. Annu Rev Biochem. 1997; 66:1-18.

28. Golemis E A, Serebriiskii I, Law S F. The yeast two-hybrid system: criteria for detecting physiologically significant protein-protein interactions. Curr Issues Mol Biol. 1999; 1:31-45.

29. Rose Z B, Dube S. Rates of phosphorylation and dephosphorylation of phosphoglycerate mutase and bisphosphoglycerate synthase. J Biol Chem. 1976; 251:4817-4822.

30. Promega corporation, 1998. Erase-a-base technical manual. URL: http://www.promega.com/tbs/tm006/tm006.pdf

31. Van Lint, J H. 1999. Introduction to Coding Theory, 3rd edition, Springer-Verlag.

32. Pope L H, Davies M C, Laughton C A, Roberts C J, Tendler S J, Williams P M. Force-induced melting of a short DNA double helix. Eur Biophys J. 2001; 30:53-62.

33. Ouyang Q, Kaplan P D, Liu S, Libchaber A. DNA solution of the maximal clique problem, Science, 1997; 278:446-449.

What is claimed is:

1. A molecular entity comprising a polypeptide core attached to at least two input nucleic acid sequences and at least one output nucleic acid sequence, wherein hybridization of said at least two input nucleic acid sequences with two complementary nucleic acid sequences modifies said at least one output nucleic acid sequence and optionally said polypeptide core.

2. The molecular entity of claim 1, wherein said polypeptide core comprises at least two catalytic functions.

3. The molecular entity of claim 2, wherein said at least two catalytic functions comprise a kinase activity and an exonuclease activity.

4. The molecular entity of claim 1, wherein said polypeptide core comprises at least one monomer of at least one dimerizable polypeptide.

5. The molecular entity of claim 2, wherein at least one of said at least two catalytic functions comprises a kinase activity.

6. The molecular entity of claim 1, capable of forming a logic gate.

7. The molecular entity of claim 6, wherein said logic gate is a NAND gate.

8. The composition-of-matter of claim 6, wherein said logic gate is a NOR gate.

9. The molecular entity of claim 6, wherein said logic gate is selected from the group consisting of the AND gate, the OR gate, the NOT gate, the XOR gate and XNOR.

10. The molecular entity of claim 5, wherein said kinase activity is a negatively regulated kinase.

11. The molecular entity of claim 10, wherein said negatively regulated kinase is DRP-1.

12. The molecular entity of claim 1, wherein said at least two input nucleic acid sequences are comprised in a single polynucleotide.

13. The molecular entity of claim 12, wherein said single polynucleotide further comprises said output nucleic acid sequence.

14. The molecular entity of claim 1, wherein each of said input nucleic acid sequences is double-stranded.

15. The molecular entity of claim 1, wherein said output nucleic acid sequence is double-stranded.

16. The molecular entity of claim 1, wherein said polypeptide core comprises a phosphatase activity.

17. The molecular entity of claim 1, wherein modification of said at least one output nucleic acid sequence comprises nucleic acid denaturation.

18. The molecular entity of claim 1, wherein modification of said polypeptide core comprises phosphorylation.

19. A composition-of-matter comprising a plurality of molecular entities, each of said plurality of molecular entities comprising a polypeptide core attached to at least two input nucleic acid sequences and at least one output nucleic acid sequence, wherein hybridization of said at least two input nucleic acid sequences with two complementary nucleic acid sequences of said plurality of molecular entities modifies said at least one output nucleic acid sequence and optionally said polypeptide core.

20. The composition-of-matter of claim 19, wherein said polypeptide core comprises at least two catalytic functions.

21. The composition-of-matter of claim 20, wherein said at least two catalytic functions comprise a kinase activity and an exonuclease activity.

22. The composition-of-matter of claim 19, wherein said polypeptide core comprises at least one monomer of at least one dimerizable polypeptide.

23. The composition-of-matter of claim 19, wherein said polypeptide core of each of said plurality of molecular entities is identical.

24. The composition-of-matter of claim 20, wherein at least one of said at least two catalytic functions comprises a kinase activity.

25. The composition-of-matter of claim 19, capable of forming a logic gate.

26. The composition-of-matter of claim 19, wherein a combination of three of said plurality of molecular entities is capable of forming a logic gate.

27. The composition-of-matter of claim 19, capable of forming at least two layers of logic gates.

28. The composition-of-matter of claim 25, wherein said logic gate is a NAND gate.

29. The composition-of-matter of claim 25, wherein said logic gate is a NOR gate.

30. The composition-of-matter of claim 25, wherein said logic gate is selected from the group consisting of the AND gate, the OR gate, the NOT gate, the XOR gate and XNOR.

31. The composition-of-matter of claim 24, wherein said kinase activity is a negatively regulated kinase.

32. The composition-of-matter of claim 31, wherein said negatively regulated kinase is DRP-1.

33. The composition-of-matter of claim 19, wherein said at least two input nucleic acid sequences are comprised in a single polynucleotide.

34. The composition-of-matter of claim 33, wherein said single polynucleotide further comprises said output nucleic acid sequence.

35. The composition-of-matter of claim 19, wherein each of said input nucleic acid sequences is double-stranded.

36. The composition-of-matter of claim 19, wherein said output nucleic acid sequence is double-stranded.

37. The composition-of-matter of claim 19, further comprising ATP.

38. The composition-of-matter of claim 19, wherein said polypeptide core comprises a phosphatase activity.

39. The composition-of-matter of claim 19, wherein modification of said at least one output nucleic acid sequence comprises nucleic acid denaturation.

40. The composition-of-matter of claim 19, wherein modification of said polypeptide core comprises phosphorylation.

41. A device comprising logic gates composed of the composition-of-matter of claim 19.

42. A molecular entity comprising a polypeptide core attached to at least two nucleic acid sequences, wherein hybridization of said at least two nucleic acid sequences with two complementary nucleic acid sequences modifies said polypeptide core.
